(19) United States
(12) Patent Application Publication
(10) Pub. No.: US 2015/0175951 A1
(43) Pub. Date: Jun. 25, 2015

(54) VERTICAL MIXING BIOREACTOR AND DRIVE SYSTEM THEREFOR

(71) Applicant: PBS Biotech, Inc., Camarillo, CA (US)

(72) Inventor: C. Brian Lee, Thousand Oaks, CA (US)

(21) Appl. No.: 14/580,032

(22) Filed: Dec. 22, 2014

Related U.S. Application Data
(60) Provisional application No. 61/919,596, filed on Dec. 20, 2013.

(51) Int. Cl. C12M 1/00; C12M 1/34; C12M 1/26; C12M 1/06

(52) U.S. Cl. CPC C12M 23/28; C12M 27/06; C12M 41/42; C12M 33/04

(57) ABSTRACT

A bioreactor includes a vessel containing a fluid to be mixed and at least one mixing device driven by a non-contaminating, indirect drive system. The mixing speed of the mixing device inside of the bioreactor may be controlled by the rotating speed of the magnets or the frequency of polarity changes of electromagnets outside the vessel acting upon magnets or ferrous material in or on the impeller wheel. The impeller wheel has a plurality of radially-oriented outer paddles with gaps therebetween that mixes fluid and culture cells around the vessel. A pair of curved axial flow vanes toward the middle of the impeller wheel washes the culture mix further.

### RELATED APPLICATION INFORMATION

This patent claims priority from the following provisional patent applications: Provisional Patent Application No. 61/919,596, entitled VERTICAL MIXING BIOREACTOR AND DRIVE SYSTEM THEREFOR, filed Dec. 20, 2013.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### FIELD OF THE INVENTION

This disclosure relates to bioreactors and, more particularly, to a non-contaminating, indirect drive system for a bioreactor having a vertical impeller wheel.

### BACKGROUND OF THE DISCLOSURE

Efforts of biopharmaceutical companies to discover new biological drugs have increased exponentially during the past two decades. Bioreactors have been used for cultivation of microbial organisms for production of various biological or chemical products in the pharmaceutical, biotechnological, and beverage industry. Most biological drugs are produced by cell culture or microbial fermentation processes which require sterile bioreactors and an aseptic culture environment.

A production bioreactor contains culture medium in a sterile environment that provides various nutrients required to support growth of the biological agents of interest. Stainless steel tanks with horizontal stirring mechanisms have long been the only option for large scale production of biological products in suspension culture. Manufacturing facilities with conventional stainless bioreactors, however, face numerous problems such as large capital investments for construction, high maintenance costs, long lead times, and inflexibilities for changes in manufacturing schedules and production capacities. Such bioreactors can only be reused for the next batch of biological agents after cleaning and sterilization of the vessel. These procedures require a significant amount of time and resources, especially to monitor and to validate each cleaning step prior to reuse for production of biopharmaceutical products.

The cell therapy and vaccine & gene therapy markets mainly utilize anchorage-dependent cells that require a solid surface, such as a flat plastic plate, to grow on. By stacking dozens of plates on top of each other, this “2D” approach can achieve a certain level of scale-up that is adequate for research or clinical stage production. However, scaling up to commercial manufacturing becomes operationally and economically unfeasible, as it would require thousands of plates. A different “3D” process, where cells grow on spherical, plastic microcarriers suspended in liquid, is considered to be the only possible option for scaling up growth of anchorage-dependent cells to meet larger manufacturing requirements. Ideally, this 3D platform would also allow for scaled-down, small volume models to be representative of the culture environments in larger volumes. Thus, new cell culture processes for these emerging markets could be developed efficiently and economically at the laboratory scale and then easily transferred to manufacturing.

A potential 3D biomanufacturing platform is the bioreactor, a controlled environment incubator for growing cells that will either be the desired product themselves or produce specific drugs/proteins. Starting in a small volume bioreactor, cells multiply in culture media until they reach critical density and then are transferred to a larger volume bioreactor; this step is repeated as necessary until the desired yield is achieved. A full suite of bioreactors in a series of increasing sizes is necessary for most biomanufacturing processes.

Stainless steel bioreactors have been the standard platform for decades in the therapeutic protein sector but have several disadvantages such as laborious sterilization steps between batches, high capital and operational costs, long-lead times to install, and large space and infrastructure requirements. Furthermore, their propeller-style impellers need to spin very quickly to mix larger volumes of liquid, resulting in increased levels of shear stress on suspended cells. The ability of anchorage-dependent cells to attach to microcarriers is presumably hindered by shear stress, which can negatively impact cell viability and result in lower product yield.

Due to the high cost of construction, maintenance, and operation of conventional bioreactors, single-use bioreactor systems made of a presterilized, disposable plastic bag or vessel fitted into a rigid metal housing have become an attractive alternative. Single-use bioreactor system vessels are replaced between individual runs, provide more flexibility on biological product manufacturing capacity and scheduling, avoid the risk of a major upfront capital investment, and simplify the regulatory compliance requirements by eliminating the need for cleaning and steam sterilization steps between batches. However, these first generation single-use bioreactors mimic the impeller-based mixing style of stainless steel, resulting in similar issues with shear stress.

Thus the majority of currently available stirred-type bioreactors have increasing levels of shear stress as volume increases, which means that small-scale models are not representative of larger culture environments. This poses serious challenges in their ability to be a low shear 3D platform for development of cell culture processes for the emerging cell therapy and vaccine & gene therapy markets. These markets are currently seeking a bioreactor platform that can successfully scale up, all the way from research and development to commercial manufacturing levels.

Achieving homogeneous fluid mixing is essential in a bioreactor or related bioproces sing equipment, where cells must be kept in suspension and mixed uniformly in the culture broth to ensure they are each exposed to similar level of gas, temperature, and nutrient composition inside the vessel. Maintaining uniform mixing helps promote a majority of the cell population, which ensures that cell growth rate and protein productivity are both predictable and consistent. This benefit applies to the traditional bioprocessing industry focused on protein or vaccine production, as well as to the emerging field of cell therapy involving stem cells and regenerative medicine. The emphasis in bioreactors for cell therapy is not only on maximizing cell number but also a maintaining a homogeneous population of certain cell identity.

Conventional bioreactor or bioprocessing vessels are cylindrical in shape with a circular top and bottom walls, a design derived from traditional chemical reactors, which allows pressurization during steam sterilization. Mixing is achieved by using an impeller or a combination of impellers that is mounted on a vertically-oriented axle or shaft, powered by a motor or magnets. The impeller has blades configured to move the fluid within the vessel predominantly up or down as it rotates horizontally. Baffles may be added to the inside of the vessel to help break up the streamlined flow of fluid around the impeller and improve mixing. The tall and narrow vessel design makes it difficult to eliminate gradients of kinetic energy dissipation within the vessel and ensure uniformly suspended solid particles such as cells and micro-carrier beads. The designs thus have limitations to achieve fast, efficient, and homogeneous fluid mixing. Problems with single-use bioreactor systems include particle settling due to the often uneven fit of heat-sealed bags on the vertical cylinder housing bottom, as well as particles becoming embedded in seams along heat sealed edges of the bag that define crevices.

In addition, powering the impeller shaft directly creates the need for a seal/bearing assembly to ensure the shaft rotates smoothly without compromising the sterility of the tank, which is critical for cultivating cells of interest without contamination. To circumvent this problem, especially in single-use systems where plastic film joining the bearing may tear at higher power rotation speeds, some bioreactors utilize non-contact drive systems such as magnetic stirring rods at the bottom of the vessel or a horizontal impeller that is magnetically-coupled to a rotating disk beneath the vessel. However, the challenges to achieve efficient vertical mixing and homogeneous suspension of micro-carrier beads or cell clumps by using a horizontal impeller in a cylindrical shaped single-use bioreactor without baffles is only worsened with this design due to the low position of the impeller, not to mention the higher likelihood of the micro-carrier beads or cell clumps become embedded into the crevices between the impeller wheel and vessel bottom, which may cause them to be ground into small particles.

Due to the aforementioned problems, there is a need for an improved drive system using an indirect rotation mechanism in order to achieve efficient, homogeneous mixing with good particle suspension in a single-use bioreactor that addresses the aforementioned deficiencies while maintaining minimum risk of contamination.

### SUMMARY OF THE INVENTION

The present application discloses various embodiments that address the drawbacks of prior bioreactor systems. In particular, the bioreactors and drive systems described herein allow for improved vertical mixing that promotes uniform liquid mixing and solid particle such as micro-carriers or cell clump suspension. Furthermore, the orientation and position of the axle and the impeller hub minimize likelihood of particles becoming embedded in crevices of wrinkled plastic film at the bottom of vessel or between the axle and the hub.

In accordance with one aspect, a bioreactor vessel contains an impeller wheel oriented on a horizontally-positioned axle, with a plurality of blades on the periphery of the impeller wheel to induce a tangential flow of the fluid in the vertical plane as it rotates. The vessel has a round bottom with smooth surface below the impeller wheel that curves upwards into a U-shape and mimics the curvature of the impeller wheel to create strong sweeping liquid flow that prevents particle settling at the bottom. Additionally, the impeller wheel contains rigid vanes in an inner portion that creates axial mixing in the horizontal plane as it rotates to complement the mixing of the vertical plane created by the blades on the impeller wheel. The fluid dynamic flow patterns combined the tangential flow on the vertical plane and the axial flow generated by internal vanes of the vertical impeller wheel also help eliminate the gradients that exist in traditional bioreactor vessels.

The size of the impeller wheel and distance from the bottom of the vessel may be varied depending on the application, but generally a larger impeller wheel with a shorter gap distance helps mix the fluid faster and prevent particle settling. The impeller wheel size and the gap distance may be increased with larger bioreactor sizes to maintain constant shear stress that the impeller wheel imparts on the cells, which helps overcome one of the major challenges with existing systems when scaling up processes to larger volumes.

In one embodiment, the impeller wheel axle remains fixed and stationary with respect to the vessel while the impeller wheel rotates freely around it. The axle is fixed to the inside wall of the vessel by means of welding or heat sealing. This allows the axle and impeller wheel to be fully contained in the vessel and obviates the need for a seal/bearing assembly that may create a leak path and compromise sterility of the vessel.

The drive systems disclosed herein utilize an indirect power input for mixing by coupling of external mechanical force with inside mixing mechanism using magnetic forces. The magnetic forces can be generated by either static magnetic coupling or electromagnetic pulsing methods. External permanent magnets or electromagnets either attract or repel the plurality of magnets positioned on the impeller wheel to drive its rotation.

A preferred aspect of disclosed herein is a bioreactor comprising a single-use vessel sized to contain a volume of fluid or medium. A vertically-oriented impeller wheel is positioned within the vessel and has a horizontally-oriented axle supported within the vessel about which the impeller rotates. The impeller wheel has radially-oriented outer paddles for mixing the fluid in the vessel with gaps therebetween and at least one curved axial flow vane located radially inward of the outer paddles. A plurality of driven elements are mounted on the impeller wheel in close proximity with an outer periphery. A drive system positioned outside of the vessel in close proximity therewith has at least one drive element configured to exert either an attractive or repulsive magnetic force to the driven element and rotate the impeller wheel. The vessel may be a disposable plastic bag or rigid vessel. There are preferably two of the curved axial flow vanes curved to impel fluid axially in opposite directions. The drive elements and driven elements may be permanent magnets, or one or the other may be ferrous elements or electromagnets. The driven elements are desirably located on a rear face of the impeller wheel and the drive system is positioned outside of the vessel adjacent the rear face of the impeller wheel, and at least one front magnet is located on a front face of the impeller wheel and an RPM sensor mounted to the vessel adjacent the front face of the impeller wheel that senses rotation of the front magnet front magnet.

### DESCRIPTION OF THE DRAWINGS

FIG. 1 is a perspective view of an exemplary bioreactor of the present application having an impeller wheel driven by a non-contact, indirect drive system;

FIG. 2 is a perspective view of a single-use disposable bioreactor vessel for use in the bioreactor of FIG. 1 illustrating an exemplary non-contact, indirect drive system positioned behind a rear wall;

FIGS. 3A and 3B are schematic front and side views of another exemplary non-contact, indirect drive system for the bioreactor impeller wheels disclosed herein;

FIGS. 4A and 4B are schematic front and side views of a further non-contact, indirect impeller drive system of the present application; and

FIGS. 5A and 5B are front and rear perspective views of an exemplary impeller wheel for use with the bioreactors disclosed herein.

FIGS. 6A-6D are perspective, side and end views, respectively, of another exemplary impeller wheel for use with the bioreactors disclosed herein.

Throughout this description, elements appearing in figures are assigned three-digit reference designators, where the most significant digit is the figure number where the element is introduced and the two least significant digits are specific to the element. An element that is not described in conjunction with a figure may be presumed to have the same characteristics and function as a previously-described element having the same reference designator.

### DETAILED DESCRIPTION

The present application provides a solution for driving bioreactor impellers that reduces contamination and maintenance. The solution involves an indirect drive system and modified impeller. In this context, “indirect” means that the impeller is driven via means other than directly rotating it through a shaft, for example. This eliminates the contamination issue with shaft seals and the like. However, pneumatic drive systems, such as disclosed in U.S. Patent Publication No. 2011/0003366 to Zeikus, are not considered indirect even though there is no shaft because of the introduction of air into the bioreactor vessel to rotate the impeller. Indirect thus means influencing the impeller completely from outside of the vessel.

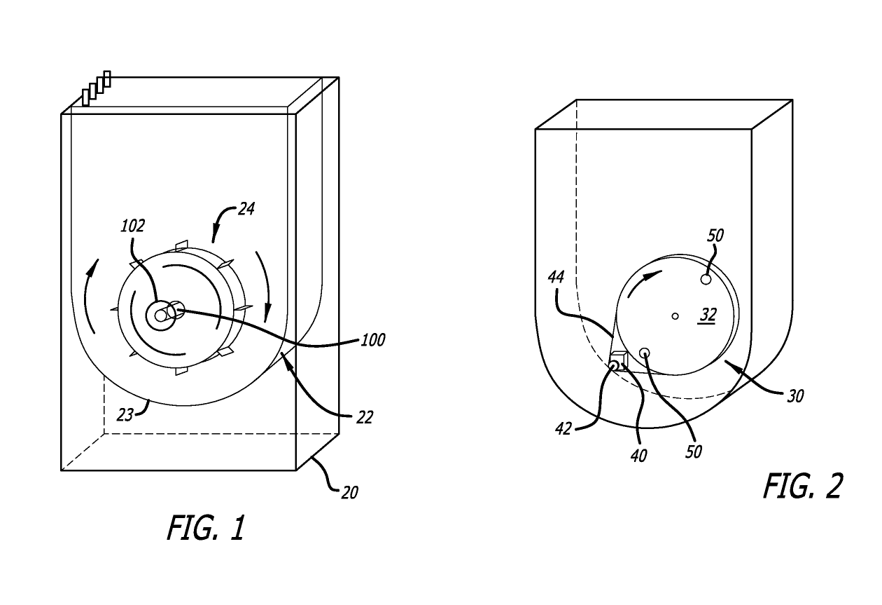

A bioreactor 20 illustrated in FIG. 1 includes a rigid outer container 20 that receives a single-use bioreactor vessel 22 of sufficient size to contain a fluid to be mixed. A variety of different sizes of bioreactors are used from the maximum working volume of 3 L up to 500 L that can use various liquid volume in each vessel. The vessel 22 is preferably a disposable bag usually made of a three-layer plastic foil, such as polyethylene terephthalate, although a rigid bioreactor vessel may also be used. Although not shown, the vessel 22 may be inserted through a top opening or front door of the outer container 20 and secured therein. Typically, there is very little space between the vessel 22 and the surrounding rigid container 20 so that when filled with fluid the flexible bag expands outward into contact with the rigid walls of the container. More particularly, the bioreactor vessel 22 comprises a generally semi-cylindrical bottom wall 23 defining a semi-cylindrical concavity therewithin. A rotatable impeller wheel 24 mounts within for rotation about a horizontal axis approximately coincident with an axis of curvature of the semi-cylindrical bottom wall, and has a rotational diameter that extends into close proximity with the semi-cylindrical bottom wall 23.

Although not shown, the rigid outer container 20 may supply a curved structural support underneath the bottom wall 23 of the vessel 22. Relevant details of such bioreactors may be found in U.S. Patent Publication No. 2011/0003366 to Zeikus, the contents of which are hereby expressly incorporated herein. The rigid outer container 20 for supporting the single-use vessel or plastic bag 22 serves several purposes, such as: providing mechanical structure for the single-use bag during a bioprocess run; protecting against physical damage to the single-use bag during a bioprocess run; allowing the end user to perform a point-of-use bag integrity testing prior to starting a run; and housing the controller system which enables full bioreactor process control. Furthermore, the outer container 20 and/or the vessel 22 provides mounts for sensors (e.g., temperature, pH, etc.) that may be inserted into the single-use vessel or bag for in-process measurements.

An exemplary non-contact drive system 30 is shown in FIG. 2 in a position behind the vessel 22. The vessel 22 is typically transparent plastic such that the drive system 30 can easily be seen through it with the impeller wheel 24 removed. More specifically, the drive system 30 includes a plate 32 rotatable about a shaft 34 that coincides with the rotational axis of the impeller wheel 24. The plate 32 may be rotated in a variety of ways, including by using a motor 40 to turn a small pinion wheel 42 around which a belt 44 is wrapped. The belt 44 is also in intimate contact with and thus rotates the outer edge of the plate 32. In this way, a speed reduction is accomplished because of the different sizes of the rotating elements.

The plate 32 includes one or more magnetic or ferrous elements 50. The elements 50 are shown as small discs and can be termed drive elements. As will be explained below, the impeller wheel 24 also includes one or more magnetic or ferrous elements (driven elements) positioned on its rear wall that attract the drive elements 50. It will thus be apparent that rotation of the plate 32 and drive elements 50 rotates the impeller wheel 24 by interaction between the drive and driven elements.

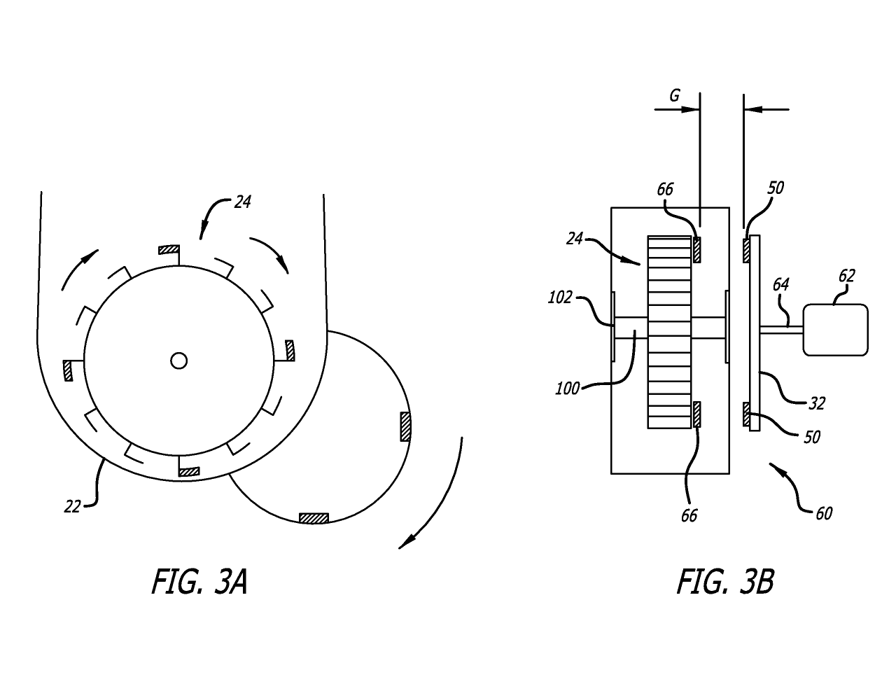

FIGS. 3A and 3B are schematic front and side views of the single-use disposable bioreactor vessel 22 for use in the bioreactor of FIG. 1, illustrating another non-contact drive system 60 positioned behind a rear wall of the vessel. The drive system 60 is similar to that described above, and thus like elements will be given like numerals. More particularly, the drive system 60 includes the aforementioned rotating plate 32 having drive elements 50 mounted thereon. Instead of a pinion wheel and belt drive, the drive system 60 features a motor 62 and a shaft 64 which directly rotates the plate 32. The drive elements 50 are shown across a gap G from a plurality of driven elements 66 mounted on the impeller wheel 24. In this embodiment, the driven elements 66 comprise magnetic or ferrous discs that are mounted on a rear wall of the impeller wheel 24 facing rearward toward the drive system 60. The gap G enables the drive system 60 to be positioned on the outside of the vessel 22.

In an exemplary embodiment, the impeller wheel diameter for a 3L vessel is 13.5 cm, and the gap G from the driven elements on the impeller wheel to the driven elements is 4 cm.

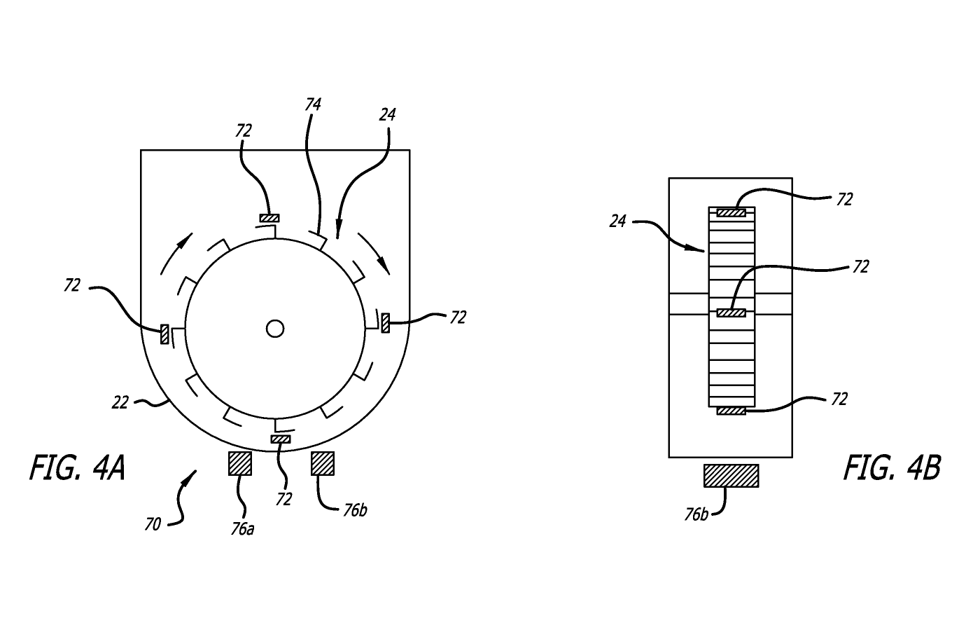

FIGS. 4A and 4B are schematic front and side views of another exemplary non-contact drive system 70 for the bioreactor impeller wheels disclosed herein. In this configuration, driven elements 72 such as magnets or ferrous pieces are mounted on a radially outer edge of the impeller wheel 24. For example, small magnetic bars 72 may be adhered to outer paddles 74 on the impeller wheel 24. Outside of and underneath the bioreactor vessel 22 are positioned at least two electromagnetics 76a, 76b spaced from one another. In the illustrated embodiment, the two electromagnets 76a, 76b are spaced on either side of a vertical mid-plane through the vessel 22. By sequentially energizing the electromagnets 76a, 76b, alternating attractive and repulsive forces can be imparted to the driven elements 72 on the impeller wheel 24.

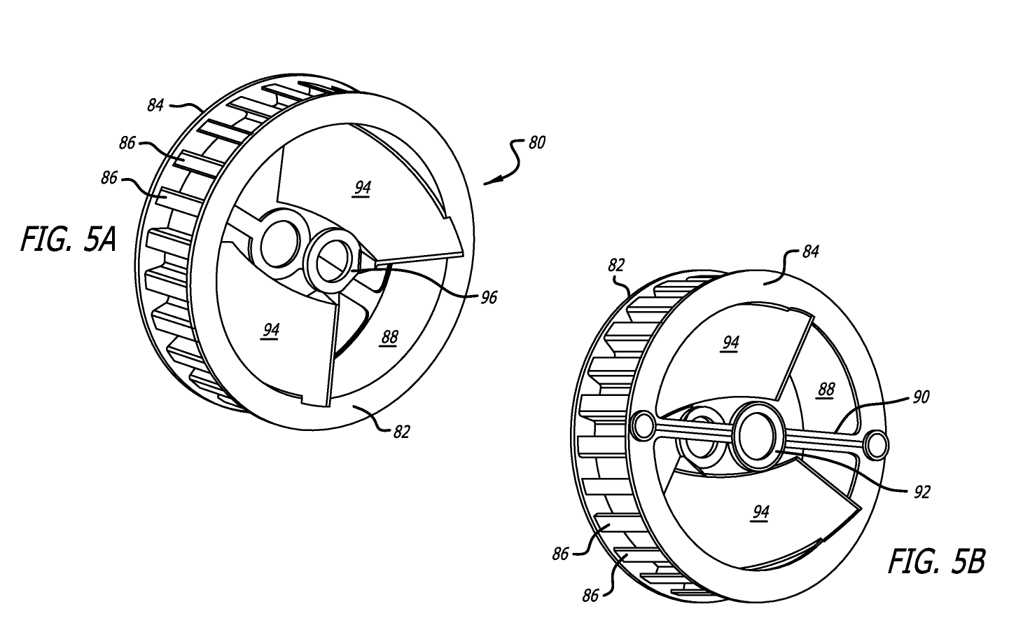

FIGS. 5A and 5B illustrate an exemplary impeller wheel 80 for use with the bioreactors disclosed herein. The impeller wheel 80 comprises a front rim 82 spaced axially from a rear rim 84 and having a plurality of rigid paddles 86 extended therebetween. A tubular wall 88 extends between the front and rear rims 82, 84 and just radially inside of the paddles 86 and defines within an inner cavity of the impeller. A diametric spar 90 extends across the inner cavity between opposite sides of the rear rim 84 and has an annular hub 92 at its center point. A pair of large curved axial flow vanes 94 are rigidly secured to the inner surface of the tubular wall 88 and project into the central cavity. As seen in FIG. 5A, a second annular hub 96 is fixed between the axial flow vanes 94 along the central axis of the impeller wheel 80. Although not shown, the annular hubs 92, 96 may be mounted to an axle that remains fixed and stationary with respect to the vessel while the impeller wheel rotates freely around it via a bearing.

With reference back to FIG. 1 and also FIG. 3B, an impeller axle 100 is fixed to the inside wall of the vessel 22 by means of a flange 102 that is welded, adhered or otherwise affixed to the inside of the vessel. A second flange (not shown) is provided on the opposite side of the impeller 24 secured to the opposite wall of the vessel 22. Because the outer container 20 is rigid, and the fit between the container and the vessel 22 is tight, the two flanges 102 are stabilized. This allows the axle 100 and impeller wheel 24 to be fully contained in the vessel and obviates the need for a mechanical seal/bearing assembly to the exterior that may create a leak path and compromise sterility of the vessel.

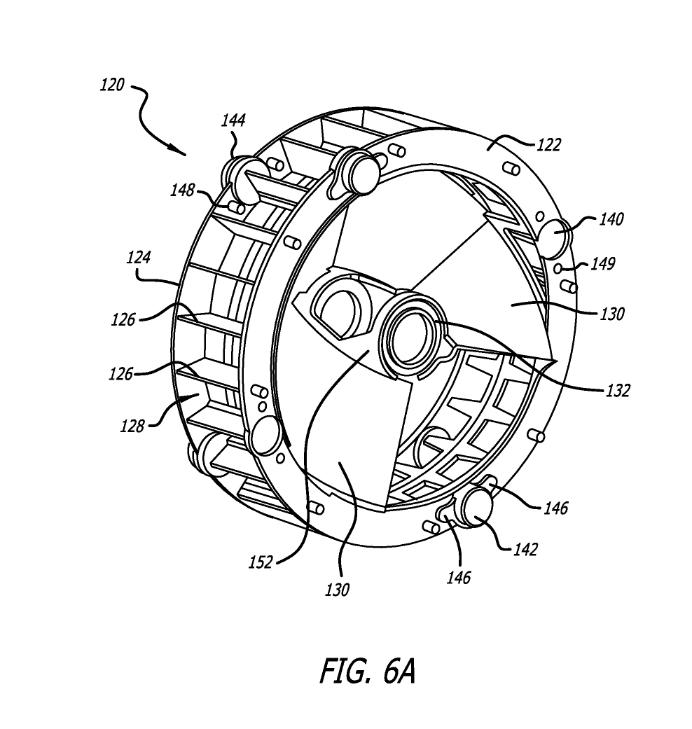

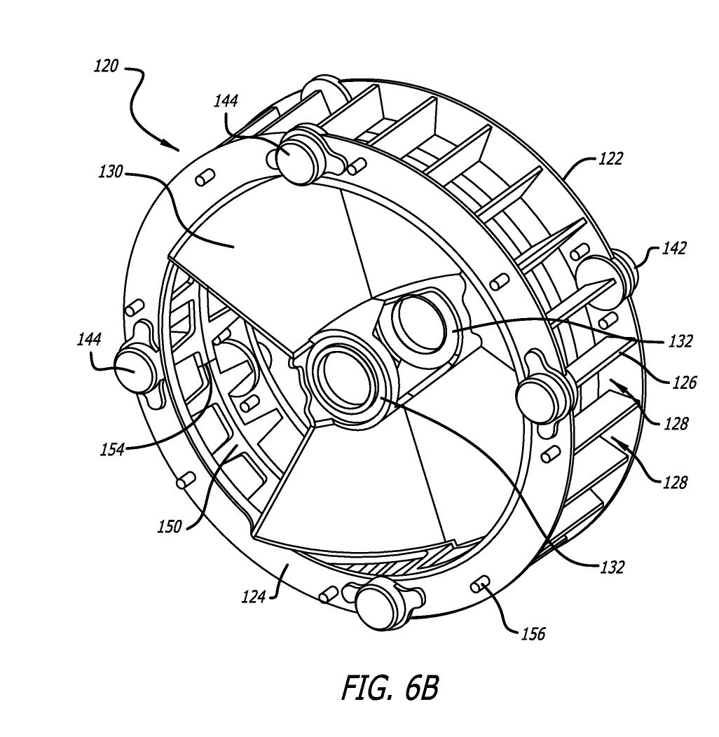

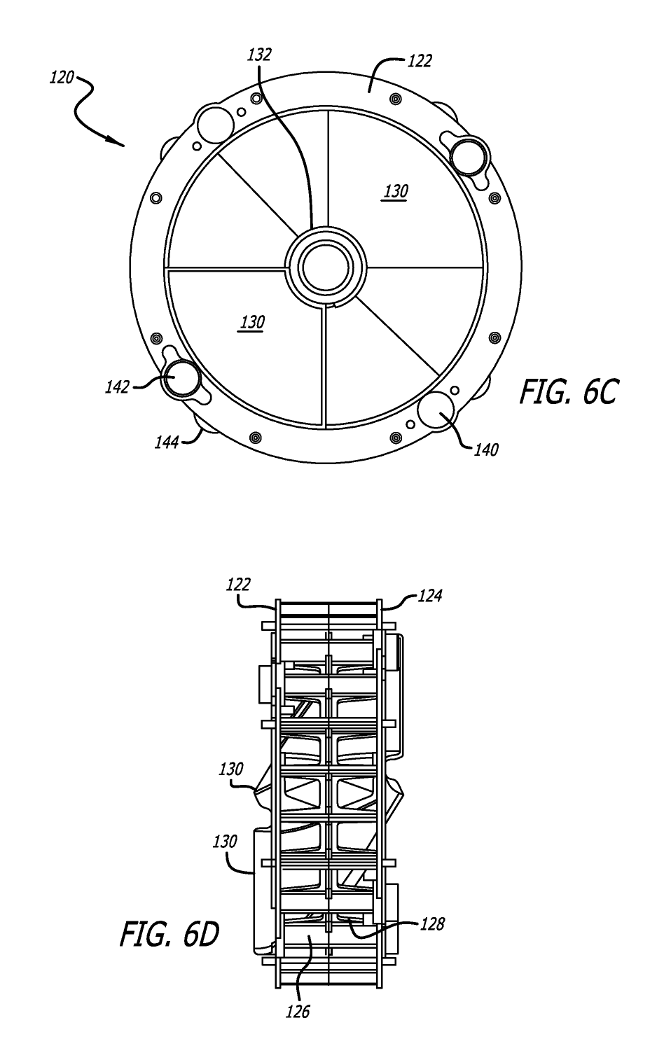

FIGS. 6A-6D illustrate several views of another exemplary impeller wheel 120 for use with the bioreactors disclosed herein. The impeller wheel 120 comprises a circular front rim 122 spaced axially from a circular rear rim 124 and a plurality of rigid radially-oriented paddles 126 extended therebetween. It should be noted that although the rims 122, 124 are circular, and thus the “wheel” is also, various other shapes other than circular (e.g., triangular, hexagonal, octagonal, etc.) may be used, and the term “wheel” encompasses more than just a circular outer shape. Gaps 128 between the paddles 126 lead to an inner cavity of the impeller wheel 120. A pair of large, curved axial flow vanes 130 are rigidly secured to the inner surface of the rims 122, 124 and project into the central cavity. A pair of spaced apart annular hubs 132 are fixed between the axial flow vanes 130 along the central axis of the impeller wheel 120. As described above with respect to FIGS. 1 and 5A-5B, the annular hubs 136 mount to an axle that remains fixed and stationary with respect to the vessel while the impeller wheel 120 rotates freely around it via a bearing. With sufficient clearance, the impeller wheel 120 may rotate about the axle without a bearing.

FIG. 6A shows a front side of the impeller wheel 120, while FIG. 6B shows a rear side. Both the front and rear rims 122, 124 have formed therein a plurality of mounting holes 140 for securing magnet assemblies 142 therein. In the illustrated embodiment, there are four large mounting holes 140 angularly spaced apart 90° around each of the rims 122, 124, although more or less than four holes may be provided. The front rim 122 of the impeller wheel 120 has two magnet assemblies 142 mounted in two diametrically-opposed holes 140, while the other two holes remain empty. Conversely, the rear side rim 124 has four magnet assemblies 144 in each of the mounting holes 140. The magnet assemblies 142, 144 typically comprise cylindrical plastic (non-magnetic) housings containing disk-shaped magnets therein for sterility. A pair of wing flanges 146 extend in opposite directions from each of the housings and preferably include small threaded studs 148 which may be secured in fastener holes 149 (see empty mounting holes 140 in FIG. 6A) provided on each side of the mounting holes 140. The front magnet assemblies 142 are shown slightly rotationally offset from the rear magnet assemblies 144, such as seen in FIG. 6C. This is merely for the sake of clarity, to visually distinguish the two sets of magnets 142, 144, and the magnets can be aligned as well.

The front magnet assemblies 142 are provided for RPM sensing of the speed of the impeller 120, while the rear magnet assemblies 144 are used with a non-contact drive system, such as described above. That is, a drive system such as the non-contact drive system 30 shown in FIG. 2 is positioned outside into the rear of a bioreactor vessel that houses the impeller wheel 120 in close proximity therewith. The drive system has at least one drive element configured to exert either an attractive or repulsive magnetic force to the driven magnet assemblies 144 and rotate the impeller 120. In this regard, the drive elements and driven magnet assemblies 144 may comprise magnetic or ferrous elements. The driven magnet assemblies 144 can be termed to the elements. Rotation of the drive system thus rotates the impeller wheel 120 by interaction between the drive and driven elements.

In a preferred embodiment, there are four driven magnet assemblies 144 spaced evenly about the rear rim 124. For maximum torque, the drive system has corresponding drive elements; that is four magnets spaced evenly about a drive plate or wheel (such as drive wheel 32 shown in FIG. 2). Because of the lack of contact between the drive system and the impeller wheel 120, there may be some slippage. Therefore, it is important to monitor the actual rotational speed and rotations of the impeller wheel 120. The front magnet assemblies 142 interact with a sensor mounted to the front of the bioreactor vessel in which the impeller wheel 120 rotates. Although a single magnet assembly 142 may be used, two spaced 180° apart our preferred so that the RPM sensing unit can determine the speed of the impeller wheel 120 that much quicker. The axial spacing of the front and rear magnet assemblies 142, 144 and placement of the RPM sensor on the front side of the reactor vessel ensure that the RPM sensor reliably and accurately measures the actual speed of the impeller wheel 120 and not the speed of the drive system magnets. Preferably, the RPM sensor provides accurate” speed measurements of within ±1 RPM.

The impeller wheel 120 provides excellent stifling action in the vertically-oriented bioreactors described herein. First of all, the radially-oriented paddles 126 extend outward to the radial edge of the rims 122, 124, and thus come into close contact with a generally semi-cylindrical bottom wall of the bioreactor vessel in which it rotates, such as described above. As before, impeller wheel 120 rotates about a horizontal axis approximately coincident with an axis of curvature of the semi-cylindrical bottom wall, and has a rotational diameter that extends into close proximity with the semi-cylindrical bottom wall such that the radial paddles 126 sweep the cells being cultured within the vessel from the bottom to the top of the liquid column. This is important for homogeneous fluid mixing, where cells must be kept in suspension and mixed uniformly in the culture broth to ensure they are each exposed to similar level of gas, temperature, and nutrient composition inside the vessel. Furthermore, the gaps 128 between the paddles 126 permit the fluid being next to travel radially inward, whereupon the axial flow vanes 130 impel the fluid and cells being cultured axially from within the impeller wheel 120. There are two axial flow vanes 130 mounted within the impeller wheel 120 in the opposite rotational sense. That is, the vanes 130 are curved to impel fluid axially in opposite directions, to encourage better homogeneity of mixing. There are no dead-ends or otherwise closed regions in and around the impeller wheel 120 so that no particles can become trapped in a dead spot and thus prevent homogeneous mixing. In addition, micro- or macro-carriers are sometimes used to facilitate cell growth. These carriers are small plastic beads or other such elements having surfaces on which the cells grow. The relatively large gaps 128 between the paddles 126 permit flow of even the largest macro-carriers, and thus induce good mixing and homogeneous cell growth. Liquid and solid particles such as cells and microcarriers are effectively mixed and prevented from accumulating in the crevices of the impeller wheel 120.

To further facilitate mixing in the center of the impeller wheel 120, each of the hubs 132 features partial tubular shrouds 152 that help redirect the flow of the culture mix axially from within the impeller wheel. As seen in the end view of FIG. 6D, the curved axial flow vanes 130 project slightly axially outside of each of the front and rear rims 122, 124. The impeller wheel 120 as an axial dimension that fits relatively closely within the surrounding bioreactor vessel, and thus the axial flow vanes 130 help stir fluid in the side gaps between the impeller wheel and the vessel.

With reference again to FIGS. 6A and 6B, a preferred construction of the impeller wheel 120 includes a plurality of the radial paddles 126 mounted to a circular spar 150 that is equidistantly spaced between the front and rear rims 122, 124 and radially positioned approximately at the inner edge of the rim. The paddles 126 stick outward from the spar 150. For the sake of manufacturing, the spar 150 may be broken up into multiple pieces, such as seen at junction 154 in FIG. 6B. A series of axially-oriented pins 156 secured to the paddles 126 or spar 150 project through holes in each of the front and rear rims 122, 124 to secure the assembly together. The parts may be secured with fasteners or adhesives, or may be heat-welded together.

As mentioned, the driven and drive elements can be either magnetic or ferrous. It will be understood by those of skill in the art, that magnetic forces can be generated between two opposite magnets, and between a magnet and a ferrous elements. Therefore, various combinations are contemplated, though magnets are desirably used for both driven and drive elements to increase the force and thus reduce their size. In terms of magnet specifications, a suitable example is a strong rare-earth magnet composed of neodymium with nickel plating. The magnets are sealed in completely to ensure no metal corrosion that may impact cell culture. The current grade of neodymium magnets used are either “N42” or “N52” (the higher number denotes stronger magnet). Disk or bar magnets may be used depending on where the magnets will be positioned on the wheel impeller, and the size of the magnets is determined by the distance between the drive and driven elements. There are typically 2 magnets on each wheel impeller. For example, a 3L impeller wheel may use N52 grade disc magnets that are ⅜″ diameter×¼″ thickness, while a 15L impeller wheel uses N42 grade bar magnets that are 1″×¼″×⅛″ thickness. Alternatively, as mentioned above, eletromagnets may also be used as either or both of the drive or driven impeller elements.

An advantageous component of the disclosed single-use bioreactors is the innovative vertical-wheel mixing mechanism, which promotes homogenous liquid mixing and particle suspension with lower shear stress and minimal power input across a full range of working volumes from 0.1 to 500 liters. Due to the consistent, predictable mixing performance of vertical-wheel technology, processes developed in small-scale “bench-top” bioreactors can confidently be scaled up in larger bioreactors for commercial manufacturing. With vertical-wheel technology, the bioreactors are uniquely capable of providing the mixing performance and scalability needed by the cell therapy and vaccine & gene therapy markets.

The vertical-wheel system is commercially available with two different drive mechanisms: the presently disclosed magnetic drive and an “AirDrive” system which uses bubbles to rotate the wheel. While they both provide similar mixing characteristics, each one is optimized for particular cell culture needs. In magnetic drive bioreactors, magnetic coupling controls rotation of the vertical-wheel and the need for antifoaming agents or shear protectants is eliminated. With their unique capability for gentle particle suspension, magnetic drive bioreactors are optimal for culturing shear sensitive products for the cell therapy and vaccine & gene therapy markets. AirDrive bioreactors are optimal for high-density cell culture processes where shear stress and foaming is less of an issue, such as those needed in the therapeutic protein market. Thus customers have the flexibility to accommodate a wide array of cell culture processes, from research and development to manufacturing, in these three biological drug sectors.

Furthermore, the present bioreactors are designed for unmatched flexibility, reliability, and ease of use. The compact housing unit is plug and play right out of the box, with very simple setup and training requirement. An embedded graphical user interface (GUI) touch screen controller provides an intuitive interface for process control. With a much smaller footprints compared to competing bioreactors of similar volume, the bioreactors require much less space and equipment setup in manufacturing facilities, leading to greater operational efficiency, flexibility, and cost savings.

Closing Comments

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

1. A bioreactor, comprising:
a. a single-use vessel sized to contain a volume of fluid or medium;
b. a vertically-oriented impeller wheel positioned within the vessel and having a horizontally-oriented axle supported within the vessel about which the impeller wheel rotates, the impeller wheel having outer paddles for mixing the fluid in the vessel and at least one driven element mounted thereon in close proximity with an outer periphery; and
c. a drive system positioned outside of the vessel in close proximity therewith having at least one drive element configured to exert either an attractive or repulsive magnetic force to the driven element and rotate the impeller wheel.

2. The bioreactor of claim 1, further including:
a. a control mechanism that can adjust the speed of the drive element by either the rotational speed or frequency of magnetic polarity of the drive system, which consequently adjusts the rotational speed of the impeller wheel;
b. the vessel forms a closed system to ensure a sterile environment in order to cultivate pure culture without contamination;
c. one or more tubes securely connected to the vessel to deliver or withdraw liquid or gas.

3. The bioreactor of claim 1, wherein the vessel is a disposable plastic bag.

4. The bioreactor of claim 1, further including at least one curved axial flow vane mounted radially within the outer paddles.

5. The bioreactor of claim 1, wherein the paddles are radially-oriented and have gaps therebetween that permit fluid flow therethrough.

6. The bioreactor of claim 1, wherein the drive elements and driven elements are permanent magnets.

7. The bioreactor of claim 1, wherein the driven elements are located on a rear face of the impeller wheel and the drive system is positioned outside of the vessel adjacent the rear face of the impeller wheel, and further including at least one front magnet located on a front face of the impeller wheel and an RPM sensor mounted to the vessel adjacent the front face of the impeller wheel that senses rotation of the front magnet front magnet.

8. The bioreactor of claim 1, wherein at least one of the drive elements and driven elements is an electromagnet.

9. A bioreactor, comprising:
a. a single-use vessel sized to contain a volume of fluid or medium;
b. a vertically-oriented impeller wheel positioned within the vessel and having a horizontally-oriented axle supported within the vessel about which the impeller wheel rotates, the impeller wheel having radially-oriented outer paddles for mixing the fluid in the vessel with gaps therebetween and at least one curved axial flow vane located radially inward of the outer paddles, and a plurality of driven elements mounted thereon in close proximity with an outer periphery; and
c. a drive system positioned outside of the vessel in close proximity therewith having at least one drive element configured to exert either an attractive or repulsive magnetic force to the driven element and rotate the impeller wheel.

10. The bioreactor of claim 9, further including:
a. a control mechanism that can adjust the speed of the drive element by either the rotational speed or frequency of magnetic polarity of the drive system, which consequently adjusts the rotational speed of the impeller wheel;
b. the vessel forms a closed system to ensure a sterile environment in order to cultivate pure culture without contamination;
c. one or more tubes securely connected to the vessel to deliver or withdraw liquid or gas.

11. The bioreactor of claim 9, wherein the vessel is a disposable plastic bag.

12. The bioreactor of claim 9, wherein there are two of the curved axial flow vanes curved to impel fluid axially in opposite directions.

13. The bioreactor of claim 9, wherein the drive elements and driven elements are permanent magnets.

14. The bioreactor of claim 9, wherein the driven elements are located on a rear face of the impeller wheel and the drive system is positioned outside of the vessel adjacent the rear face of the impeller wheel, and further including at least one front magnet located on a front face of the impeller wheel and an RPM sensor mounted to the vessel adjacent the front face of the impeller wheel that senses rotation of the front magnet front magnet.

15. The bioreactor of claim 9, wherein at least one of the drive elements and driven elements is an electromagnet.
